(19) United States
(12) Patent Application Publication
(10) Pub. No.: US 2007/0242807 A1
(43) Pub. Date: Oct. 18, 2007

(54) Direct digital radiograhic combination positioning table and transforming immobilizing stand

(76) Inventor: Roy Anthony Brown, Carlsbad, CA (US)

Correspondence Address: Roy Anthony Brown, Unit A, 4629 Park Dr., Carlsbad, CA, 92008, US

(21) Appl. No.: 11/714,540

(22) Filed: Mar. 5, 2007

Related U.S. Application Data
(60) Provisional application No. 60/779,230, filed on Mar. 3, 2006.

(51) Int. Cl. A61B 6/04

(52) U.S. Cl. 378/209

(57) ABSTRACT

This invention is associated to with the practicality and method of acquiring of Radiographs of a human body. Particularly of the foot when the patient is standing utilizing direct digital receptors. Presently, to obtain a weight bearing, anterior-posterior foot, you must stand on the media that you intend to record on, in this case a direct digital receptor. Table will protect the highly sensitive direct digital X ray receptor from weight of a patient. This unique feature will allow this type of equipment to produce these exams. The Table can also be modified to perform as a fixed height positioning table for routine examinations. This device is designed to “Transform” into a “Stand” to acquire images to “Stitch”, a process where two or more digital radiographs are aligned to form one image such a Scoliosis examination or Leg Length studies. The foregoing features and advantages of the present invention will be apparent from the following more particular description of the invention. The accompanying drawings, listed herein below, are useful in explaining the invention.

The invention relates to a weight-bearing apparatus (table) to position the human foot/feet in a weight bearing position. Comprising of a radiolucent transparent top for supporting the weight of a patient. With the onset of this new technology there is no present method for accomplishing this task short of standing directly on the detector. Using established methods for acquiring weight bearing, anterior-posterior (AP) projections of a patient's foot/feet are not possible. Table has a transparent, radiolucent top forming a portion of the middle surface allowing an unobstructed view of the Digital X ray receptor. This allows the foot/feet of a patient standing on tabletop to be visualized and determine proper positioning of the patient's foot to X-ray source. The table also enables the taking of lateral projections of the foot and ankle and knee by facilitating the height requirement needed to obtain these views relevant to the fixed nature of the detectors. Steps leading up to tabletop allows patient access to receptor. This safely assures access and minimal elevation of the patient from the floor, rails extend upwardly above tabletop giving support for patients as they ascend.

This invention is also associated to with the practicality and method of acquiring full-length images of the vertebral column or Scoliosis film and lower extremities or leg length studies. Presently to obtain these views the patient must stand absolutely motionless or stabilizing themselves on the detector while a combination of x ray source and digital detector are positioned around them. This is extremely difficult, as any motion resulting from fatigue or the change of positions associated with the curvatures of the human anatomy in contact with the detector will result in improper alignment of the “stitched” image. The benefits of performing these types of exams diminish, as reattempts of these images accumulate and the risk of potentially dangerous ionizing radiation to the patient becomes a danger. Table can “Transform” into a positioning “Stand”, both sides of the stand are used in the acquiring of images for the “Stitching” process where two or more digital radiographs are digitally aligned to form one seamless examination. Positioning Stand having a flat surface for supporting and immobilizing a patient. The radiolucent quality allows for the X ray beam to pass through surface and into the direct digital detector. The transparency enables the technologist to view the positioning of the direct digital receptor relative to the patient's.

This invention is also associated to with the practicality and method of acquiring all other views associated with the supine, prone and lateral positions of the human anatomy. Currently the widely used positioning table accommodates the positioning needs and demands for routine radiography. The table can be modified into a fixed height-positioning table to facilitate views of the human anatomy that require a patient to lie horizontal. Table has a transparent, radiolucent top forming a portion of the middle surface and extensions on the front and back allowing an unobstructed view of the Digital X ray receptor under 80 percent of the table's surface. The radiolucent quality allows for the X ray beam to pass through surface and into the direct digital detector. The transparency enables the technologist to view the positioning of the direct digital receptor relative to the patient's.

What is claimed is:

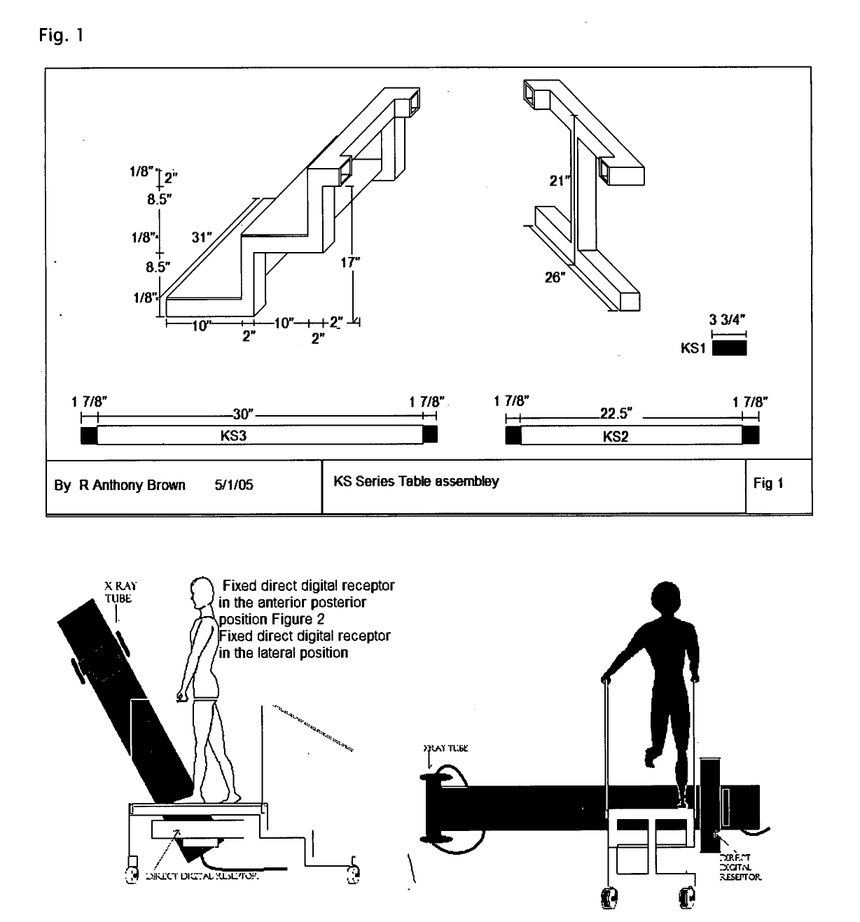

1. Table is segmented into three major components; Stairs, sides and ends to accommodate any length to perform as an accessory stair case, span and cover only the dimension of the digital receptor, oversized to provide more work area. Apparatus is constructed utilizing above stated methods wherein said apparatus is comprised of:
a) Frame that will form a table/stand to accommodate stair treads and a radiolucent transparent surface.
b) Steps that lead to the standing surface (table top)
c) Radiolucent transparent table top forming the middle surface allowing as unobstructed view of the direct digital detector/receptor.
d) Side rails comprising of two or three support or guard rail at right angle to the table top and extend upward from the edges of the table top. While in the stand position rails double as stabilization aids.
e) Wheels that swivel and lock, thus providing a mobile and secure stationary table/stand when a patient is upon it.
As seen in FIG. 1. Construction of unit is modular and interchangeable.

2. An apparatus constructed utilizing methods in claim 1 aid in the positioning of patients between an X-ray source, direct beam and into a Digital X-ray receptor to take X-rays of a weight-bearing foot, ankle and knees in the lateral position. The table permits a digital x-ray receptor to freely move alongside a standing patient. Steps leading up to tabletop, allowing for minimal elevation of the patient from the floor. Railing extending upwardly above surface gives support for patients as they ascend. Tabletop has a transparent, radiolucent surface forming the middle surface allowing an unobstructed view of the plantar (bottom) of foot as seen in FIG. 2. This allows the foot of a patient standing on tabletop to be visualized and determine proper positioning of the patient's foot to X-ray source.

3. An apparatus constructed utilizing methods in claim 1 transforms into a positioning stand for self immobilizing of patients between an X-ray source, direct beam and into a Digital X-ray receptor to take X-rays of Scoliosis or Leg Length studies for evaluation of spine and lower extremities. Adjustable hinges on the first step transforms table described in Description [Para2] and as seen in FIG. 3. The stand permits a digital x-ray receptor to freely move behind a standing patient. On the either side, steps on the leg side in the upright position allow patient access to receptor for the leg length study. This safely assures access and minimal elevation of the patient from the floor. On the Scoliosis side patient stands on the floor wile the X-ray receptor moves freely behind the standing patient. Rails extend outwardly from both sides of the stand giving support for patients as they self immobilize themselves during exam.

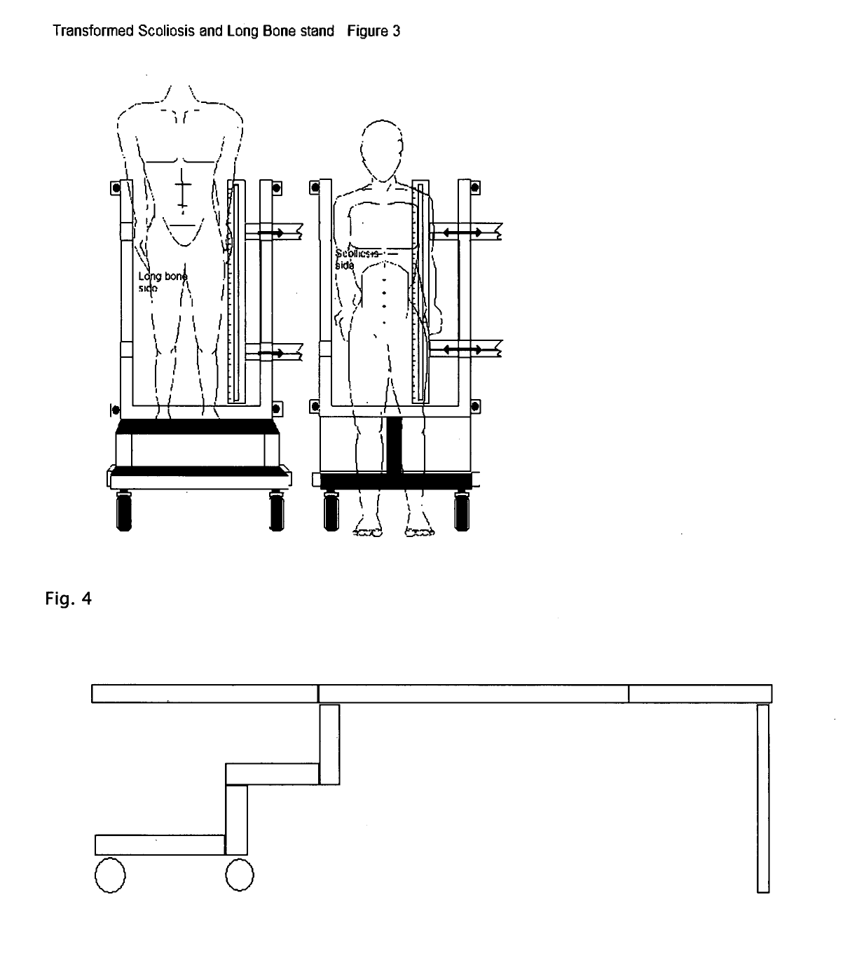

4. An apparatus constructed utilizing methods in claim 1 and described in [Para 3] and seen in FIG. 4, aid in the positioning of patients between an X-ray sources, direct beam and into a Digital X-ray receptor to take X-rays of all supine, prone and lateral positions. By removing all rails, attaching front and back extensions and moving back legs outward to the edges of the back extension allows access to 80 percent of the horizontal surface to facilitate positioning and views of the human body for radiographic purposes.
